(19) United States
(12) Patent Application Publication
(10) Pub. No.: US 2026/0194499 A1
(43) Pub. Date: Jul. 9, 2026

(54) METHOD AND SYSTEM FOR TRACKING QUALITY OF FARMED AQUATIC PRODUCTS BASED ON LIPIDOMIC FINGERPRINTING

(71) Applicant: South China Normal University, Guangzhou (CN)

(72) Inventors: Lei WANG, Guangzhou (CN); Yixiang ZHAO, Guangzhou (CN); Kaiwen YOU, Guangzhou (CN); Haitao ZHANG, Guangzhou (CN); Yongjie JIANG, Guangzhou (CN); Zhuoduo WANG, Guangzhou (CN); Limei ZHAO, Guangzhou (CN)

(73) Assignee: South China Normal University, Guangzhou (CN)

(21) Appl. No.: 19/434,022

(22) Filed: Dec. 29, 2025

(30) Foreign Application Priority Data
Jan. 6, 2025 (CN) 202510011834.0

(51) Int. Cl. G01N 30/72; G01N 30/02; G01N 30/12; G01N 30/86; G01N 33/12

(52) U.S. Cl. CPC G01N 30/7206; G01N 30/8631; G01N 30/8686; G01N 30/8696; G01N 33/12; G01N 2030/025; G01N 2030/128

(57) ABSTRACT

The present disclosure belongs to the technical field of data processing and proposes a method and system for tracking quality of farmed aquatic products based on lipidomic fingerprinting. The method specifically incudes: identifying a farmed aquatic product circulation process and selecting sampling points in the aquatic product circulation process; performing HS-SPME-based treatment on farmed aquatic products at each sampling point to obtain aquatic product volatile samples; collecting GC-MS total ion chromatogram information about the aquatic product volatile samples and constructing peak area vectors; calculating a quality attenuation density according to the peak area vector of each sampling point; and feeding back a circulation step where a risk of quality attenuation occurs to a manager or management system through the quality attenuation density.

### CROSS-REFERENCE TO RELATED APPLICATIONS

The application claims priority to Chinese patent application No. 202510011834.0, filed on Jan. 6, 2025, the entire contents of which are incorporated herein by reference.

### TECHNICAL FIELD

The present disclosure belongs to the technical field of data acquisition and aquatic product quality tracking, and specifically relates to a method and system for tracking quality of farmed aquatic products based on lipidomic fingerprinting.

### BACKGROUND

In a farmed aquatic product circulation process, ensuring the quality of farmed aquatic products is the key to circulation management. The actual steps involved in circulation management mainly include fishing, storage, transportation and display. Moreover, there are still many steps in these main steps depending on the species distinction of farmed aquatic products. The quality of farmed aquatic products described in these steps usually refers to the important sensory experience of farmed aquatic products in taste, flavor or smell. Since these sensory experience directly affect consumption choices of consumers, high-precision analysis is often performed on the quality of farmed aquatic products in the farmed aquatic product circulation process in farming management, thereby reducing the risks of quality loss of farmed aquatic products.

Currently, there are different methods for detecting the taste, flavor, or odor of farmed aquatic products. Often, one method is only used to detect one quality characteristic of the aquatic products. For example, the gas chromatography-mass spectrometry is used to detect the influence of fishy substances produced by oxidation and microbial metabolism of aquatic products on odor, texture analysis is used to detect the influence of elasticity and moisture retention capacity of muscle fibers in texture-sensitive species such as shrimp and cod on the taste, and DHS dynamic headspace analysis is used to detect the concentration and types of volatile flavor compounds released into the air by aquatic products.

However, current quality tracking of farmed aquatic products is often highly targeted, which makes the quality analysis of different varieties of farmed aquatic products in the circulation process costly and inefficient. Therefore, there is an urgent need for a method and system for tracking quality of farmed aquatic products based on lipidomic fingerprinting.

The reason for using lipidomic fingerprinting is that lipidomics covers a variety of lipid components in aquatic products. Lipidome is prone to change in an aquatic product treatment process and directly affect the taste of aquatic products, including fatty acids represented by AA (arachidonic acid), DHA and EPA, as well as components in phospholipid, triglyceride and other lipidome, which can lead to unpleasant odors and changes in taste. Aldehydes, including hexanal and heptanal, as well as ketones, produced during the peroxidation of lipidomic substances often increase fishy or off-flavors, thus significantly affecting the eating quality, which is often considered to be directly related to changes in farmed aquatic products. In addition, the degradation of phospholipid also affects the texture and flavor of the products. Different components exhibit different metabolic or degradation characteristics during the circulation process of farmed aquatic products, thus enabling tracking of quality of farmed aquatic products based on lipidomic fingerprinting.

### SUMMARY

An objective of the present disclosure is to propose a method and system for tracking quality of farmed aquatic products based on lipidomic fingerprinting, to solve one or more technical problems existing in the prior art, and at least provide a beneficial option or create condition.

In one aspect of the present disclosure, a method for tracking quality of farmed aquatic products based on lipidomic fingerprinting is provided to achieve the above objective, the method including the following steps:
S100, identifying a farmed aquatic product circulation process and selecting sampling points in the aquatic product circulation process;
S200, performing HS-SPME-based treatment on farmed aquatic products at each sampling point to obtain aquatic product volatile samples;
S300, collecting GC-MS total ion chromatogram information about the aquatic product volatile samples and constructing peak area vectors;
S400, calculating a quality attenuation density based on the peak area vector of each sampling point; and
S500, feeding back a circulation step where a risk of quality attenuation occurs to a manager or management system through the quality attenuation density.

Further, in step S100, the method for identifying the farmed aquatic product circulation process and selecting sampling points in the aquatic product circulation process includes: involving an environment detection device in the farmed aquatic product circulation process, which consists of a humidity sensors and a temperature sensor; if a temperature value measured by the temperature sensor in real time is not within the temperature range of 0-4° C., or if a humidity value measured by the humidity sensor in real time is not within the humidity range of 85-95%, defining that abnormal temperature and humidity occurs at this moment; presetting a time interval GTT between sampling points to be 10-30 minutes, if no abnormal temperature or humidity occurs within the GTT interval, taking an end moment of the GTT interval as a sampling point, otherwise, recording a time point when abnormal temperature or humidity occurs as a sampling point in the time period. That is, the time point when abnormal temperature or humidity occurs replaces the end moment of the GTT interval and is used as a sampling point in one GTT interval. There is one and only one sampling point in each GTT interval. When there are a plurality of such moments when abnormal temperature or humidity occurs, the first moment when abnormal temperature or humidity occurs is selected as a sampling point, that is, the moment when abnormal temperature or humidity is first detected is selected.

The humidity sensor is either a capacitive humidity sensor or a resistive humidity sensor, and the temperature sensor is either a thermistor sensor or a semiconductor temperature sensor.

At the fishing and initial storage stages, temperature and humidity sensors should be installed at the top, middle and bottom of a refrigerated compartment of a fishing vessel. In transportation and cold chain links, temperature and humidity sensors should be installed at the top, middle, and bottom of a refrigerated truck compartment, respectively. At the stage of displaying a refrigeration cabinet, temperature and humidity sensors are installed at the top, middle, and bottom of a worktable in a processing region. When there are a plurality of reading values from the same type of sensors, the average is taken as a real-time reading value of that type of sensors. The environment detection device sends the sampling points to a server in real time, and the server stores or uses the environmental data.

Further, in step S200, the method for performing HS-SPME-based treatment on the farmed aquatic products at each sampling point to obtain the aquatic product volatile samples includes: selecting farmed aquatic products at sampling points for sampling to obtain original samples; collecting aquatic product volatile samples from the original samples through an HS-SPME solid-phase microextraction method, where in the application of the HS-SPME technology, the extraction temperature is set between 30-60° C., and the extraction time is set between 10-30 minutes.

The farmed aquatic products are generally fish aquatic products. The principle of solid-phase microextraction is used in the method of the HS-SPME technology to extract volatile organic compounds from the headspace of aquatic products, specifically including: selecting SPME fibers of target compounds, which are one or more of fatty acids, phospholipids, and triglycerides, the SPME fibers including PDMS, CAR/PDMS, or PDMS/DVB fibers, and capturing volatile substances in the headspace of the aquatic products, the volatile substances including fatty acids, aldehydes, ketones, amino acids and derivatives thereof, the captured volatile substances being the volatile organic compounds as aquatic product volatile samples.

Further, in step S300, the method for collecting the GC-MS total ion chromatogram information about the aquatic product volatile samples and constructing the peak area vectors includes: introducing the aquatic product volatile samples into a GC-MS gas chromatography-mass spectrometry instrument to obtain a total ion chromatogram, reading each aquatic product volatile in the total ion chromatogram and denoting the aquatic product volatile as a test object, reading the peak area of each test object, and constructing the peak area of each test object in the sampling point into a vector and denoting the vector as a peak area vector.

The total ion chromatogram is a spectrogram obtained by accumulating the ion intensities of all mass-to-charge ratios detected by a mass spectrometer at each time point. A GC-MS device can accurately separate and identify different volatile compounds in a sample in the operating process. In the GC-MS process, chemical components in the sample are separated in a chromatographic column according to molecular weight, polarity and other characteristics thereof, forming a series of peaks. The mass spectrometer ionizes the separated components and performs qualitative and quantitative analysis according to the ionized mass spectrometry information.

The total ion chromatogram obtained by GC-MS provides detailed information about various chemical components in the sample, including the position of each peak, i.e. retention time and peak area. Each peak represents a specific chemical component or class of compounds. The peak area vectors are obtained by quantifying the area of each peak in the total ion chromatogram.

Further, in step S400, the method for calculating the quality attenuation density according to the peak area vector of each sampling point includes: setting a time interval formed by all sampling points as a sampling period, and defining sampling points in reverse and forward time directions of each sampling point as backward-direction points and forward-direction points thereof respectively; calculating cosine distances between peak area vectors of each sampling point and all forward-direction points thereof, and denoting the forward-direction point with the maximum cosine distance as a first marker point; denoting the average of the cosine distances of all the forward-direction points as a forward-direction cosine distance; setting a first distance condition to be that the cosine distance is greater than the forward-direction cosine distance, and setting a second distance condition to be that at least one intermediate forward-direction point having a cosine distance less than the forward-direction cosine distance exists between the sampling point and the forward-direction point; searching for a first marker point that satisfies the first and second distance conditions in reverse time order from the sampling points and denoting the first marker point as a second marker point; counting the number of backward-direction points that have the same second marker point as a current sampling point in the reverse time direction of the sampling points and denoting the number as a reverse-charging number, and presetting a reverse-charging coefficient Rc.idx, Rc.idx∈[0.3, 1]; rounding up the product of the reverse-charging number and the reverse-charging coefficient to a reverse-charging value Rc.vl, defining a third marker point of the sampling point as the Rc.vl-th forward-direction point of the second marker point in the forward time direction, and denoting a time interval between the sampling point and the third marker point as a reference domain of the sampling point; and denoting the ratio of the cosine distances between the second and third marker points as a reverse-charging regression ratio Rc.rvt of the sampling points.

The intermediate forward-direction point refers to each forward-direction point between the sampling point and the forward-direction point participating in judgment of the second distance condition. A default value of the pre-set reverse-charging coefficient is 0.5, and is used to adjust a computational model for calculating quality attenuation. When class-adding objects appear frequently in most sampling points, the value is increased, otherwise, the value is decreased, thereby enhancing the rejection of the calculated quality attenuation density and preventing overfitting.

For any sampling point, the peak area vector of the sampling point is subtracted from that of the first sampling point in the reverse time direction thereof to obtain a step difference vector, test objects corresponding to step difference elements with values greater than zero in the step difference vector are classified as class-adding objects, otherwise, are classified as class-lossing objects, and a write sequence of each class-adding object is denoted as a class-adding sequence, the step difference element referring to any element in the step difference vector; a percentile value of the step difference element of the class-adding object in a set of step difference elements corresponding to various identical class-adding objects in the reference domain is denoted as a class-adding order value, an accumulated value of class-adding order values corresponding to all class-adding objects at the sampling point is denoted as a class-adding accumulated value Icv, and forward-direction points in the reference domain of which the class-adding accumulated value is less than the class-adding accumulated value corresponding to the current sampling point are denoted as adaptive forward-direction points; and a quality attenuation density Pdt is calculated through the class-adding accumulated value and the reverse-charging regression ratio:

$$Pdt ( i 0 ) = Rc . {rvt}_{i 0} {\sum }_{i 1 = 1}^{len} exp ( {Icv}_{i 0} - {Icv}_{i 1} ) \cdot diff \{ {Pmks}_{i 0} , {Pmks}_{i 1} \} ;$$

where i0 is the serial number of the sampling point, Pdt(i0) represents the quality attenuation density of the i0^{th} sampling point, i1 is the accumulated variable, len is the number of adaptive forward-direction points corresponding to the sampling points, expo is the exponential function with the natural constant e as the base, and Icvi0 and Icvi1 are the class-adding accumulated values of the i0^{th} sampling point and the i1^{th} adaptive forward-direction point; and diff{ } is the Euclidean distance function, which returns the Euclidean distance between two calling sequences, Pmksi0 and Pmksi1 represent the class-adding reading sequences of the i0th sampling point and the i1^{th} adaptive forward-direction point respectively, and the class-adding reading sequence is composed of the class-adding sequences when the i0^{th} sampling point and the i1^{th} adaptive forward-direction point correspond to various step difference elements, where the class-adding sequence of the i1^{th} adaptive forward-direction point is the same as that of the i0^{th} sampling point.

The beneficial effects are as follows: since the quality attenuation density is an analysis result constructed according to a dynamically screened reference domain, the degree of induction of the risk of quality variation in aquatic products in subsequent circulation stages between different sampling points can be effectively quantified, this analytical method can not only clearly reflect the time points when lipidomic substances show significant changes in the aquatic product circulation process, but also accurately quantify the sustained response of these inductions to subsequent changes, thus providing a solid mathematical basis for key steps for further identifying the risk of a sudden drop in aquatic product quality.

Further, in step S500, the method for feeding back the circulation step where the risk of quality attenuation occurs to the manager or management system through the quality attenuation density includes: setting a risk management time period (RMT), RMT∈[60, 120] minutes; taking any sampling point as a current sampling point, and taking the RMT period in the reverse time direction of the current sampling point as a current risk management period; denoting the quality attenuation density of the current sampling point as Pdt, and denoting the average of various quality attenuation densities in the current risk management period as EPdt; presetting a variable risk identification threshold RPRt, a value range thereof being RPRt∈[1,1,2]; if Pdt>RPRt×EPdt, defining that the current sampling point is at the risk of quality attenuation, and that the flavor of the aquatic products deteriorates or loses, and constructing all sampling points at the risk of quality attenuation into a sequence and sending the sequence to a manager client.

Preferably, all undefined variables in the present disclosure, if not explicitly defined, can be manually set thresholds.

The present disclosure further provides a system for tracking quality of farmed aquatic products based on lipidomic fingerprinting, including: a processor, a memory, and a computer program stored in the memory and executable on the processor, where the processor, when executing the computer program, implements the steps in the method for tracking quality of farmed aquatic products based on lipidomic fingerprinting, and the system for tracking quality of farmed aquatic products based on lipidomic fingerprinting is runnable in a computing device such as a desktop computer, a laptop computer, a handheld computer, or a cloud data center. The runnable system may include, but is not limited to, a processor, a memory, and a server cluster. The processor, when executing the computer program, operates in the following system units:
an aquatic product sampling point collection unit, configured to identify a farmed aquatic product circulation process and select sampling points in the aquatic product circulation process;
an aquatic product volatile extraction unit, configured to perform HS-SPME-based treatment on farmed aquatic products at each sampling point to obtain aquatic product volatile samples;
a peak area vector construction unit, configured to collect GC-MS total ion chromatogram information about the aquatic product volatile samples and construct peak area vectors;
an attenuation density calculation unit, configured to calculate a quality attenuation density according to the peak area vector of each sampling point; and
a quality attenuation feedback unit, configured to feed back a circulation step where a risk of quality attenuation occurs to a manager or management system through the quality attenuation density.

The beneficial effects of the present disclosure are as follows: since the quality attenuation density is an analysis result constructed according to a dynamically screened reference domain, the degree of induction of the risk of quality variation in aquatic products in subsequent circulation stages between different sampling points is effectively quantified, by extracting the quality attenuation density, the dynamic changes in quality of farmed aquatic products in the circulation process are accurately quantified, a targeted means of identifying deterioration stages is provided, and a circulation step where quality variation of aquatic products occurs in the circulation process is located ultimately. The quality attenuation density calculation unit not only significantly improves the reliability of abnormal change detection, but also has good scenario adaptability through the link of quickly locating problems which is common to a variety of aquatic products. Meanwhile, the construction of an intelligent quality monitoring system is supported, providing scientific support for the digitalization and efficiency of aquatic product circulation management.

### BRIEF DESCRIPTION OF DRAWINGS

The above and other features of the present disclosure will become more apparent from the detailed description of the embodiments shown in conjunction with the accompanying drawings. In the accompanying drawings, the same reference numerals denote the same or similar elements. Obviously, the drawings described below are merely some embodiments of the present disclosure. Those having ordinary skill in the art can also obtain other drawings from these drawings without creative effort. In the drawings:

FIG. 1 shows a flowchart of a method for tracking quality of farmed aquatic products based on lipidomic fingerprinting; and

FIG. 2 shows a structural diagram of a system for tracking quality of farmed aquatic products based on lipidomic fingerprinting.

### DETAILED DESCRIPTION OF THE EMBODIMENTS

The concept, specific structure and technical effects of the present disclosure will be clearly and completely described below with reference to the embodiments and accompanying drawings, so as to fully understand the objectives, solutions and effects of the present disclosure. It should be noted that, unless otherwise specified, embodiments in the present disclosure and features in the in embodiments can be combined with each other.

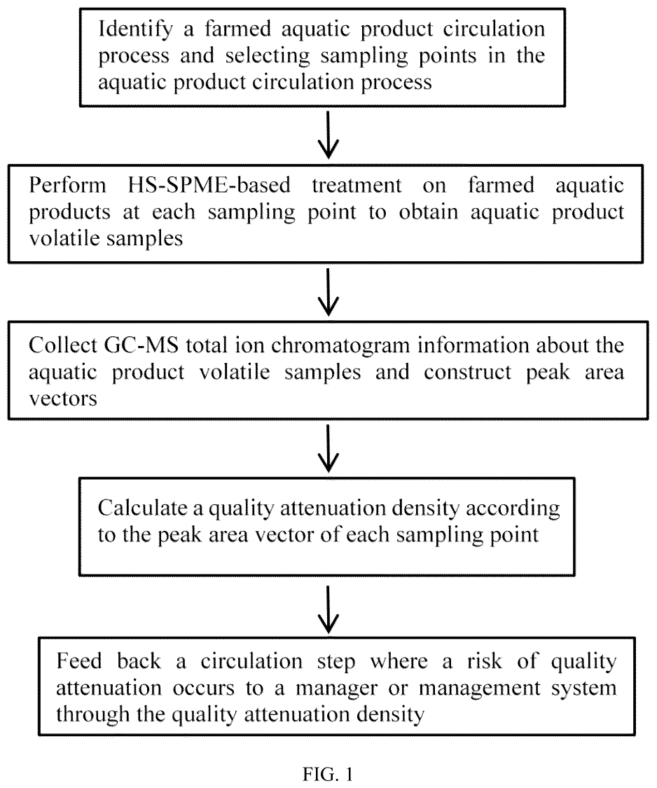

FIG. 1 shows a flowchart of a method for tracking quality of farmed aquatic products based on lipidomic fingerprinting. The following describes, with reference to FIG. 1, a method for tracking quality of farmed aquatic products based on lipidomic fingerprinting according to an embodiment of the present disclosure, the method including the following steps:
S100, identifying a farmed aquatic product circulation process and selecting sampling points in the aquatic product circulation process;
S200, performing HS-SPME-based treatment on, farmed aquatic products at each sampling point to obtain aquatic product volatile samples;
S300, collecting GC-MS total ion chromatogram information about the aquatic product volatile samples and constructing peak area vectors;
S400, calculating a quality attenuation density according to the peak area vector of each sampling point; and
S500, feeding back a circulation step where a risk of quality attenuation occurs to a manager or management system through the quality attenuation density.

Further, in step S100, the method for identifying the farmed aquatic product circulation process and selecting sampling points in the aquatic product circulation process includes: involving an environment detection device in the farmed aquatic product circulation process, which consists of a humidity sensor and a temperature sensor, if a temperature value measured by the temperature sensor in real time is not within the temperature range of 0-4° C., or if a humidity value measured by the humidity sensor in real time is not within the humidity range of 85-95%, defining that abnormal temperature and humidity occurs at this moment; presetting a time interval GTT between sampling points to be 15 minutes, if no abnormal temperature or humidity occurs within the GTT interval, taking an end moment of the GTT interval as a sampling point, otherwise, recording a time point when abnormal temperature or humidity occurs as a sampling point in the time period.

Further, in step S200, the method for performing HS-SPME-based treatment on the farmed aquatic products at each sampling point to obtain the aquatic product volatile samples includes:
selecting aquatic products at sampling points for sampling to obtain original samples; and collecting aquatic product volatile samples from the original samples through an HS-SPME solid-phase microextraction method, where in the application of the HS-SPME technology, the extraction temperature is set to 40° C. and the extraction time is set to 30 minutes.

Further, in step S300, the method for collecting the GC-MS total ion chromatogram information about the aquatic product volatile samples and constructing the peak area vectors includes: introducing the aquatic product volatile samples into a GC-MS gas chromatography-mass spectrometry instrument to obtain a total ion chromatogram, reading each aquatic product volatile in the total ion chromatogram and denoting the aquatic product volatile as a test object, reading the peak area of each test object, and constructing the peak area of each test object in the sampling point into a vector and denoting the vector as a peak area vector.

Further, in step S400, the method for calculating the quality attenuation density according to the peak area vector of each sampling point includes: setting a time interval formed by all sampling points as a sampling period, and defining sampling points in reverse and forward time directions of each sampling point as backward-direction points and forward-direction points thereof respectively; calculating cosine distances between peak area vectors of each sampling point and all forward-direction points thereof, and denoting the forward-direction point with the maximum cosine distance as a first marker point; denoting the average of the cosine distances of all the forward-direction points as a forward-direction cosine distance; setting a first distance condition to be that the cosine distance is greater than the forward-direction cosine distance, and setting a second distance condition to be that at least one intermediate forward-direction point having a cosine distance less than the forward-direction cosine distance exists between the sampling point and the forward-direction point; searching for a first marker point that satisfies the first and second distance conditions in reverse time order from the sampling points and denoting the first marker point as a second marker point; counting the number of backward-direction points that have the same second marker point as a current sampling point in the reverse time direction of the sampling points and denoting the number as a reverse-charging number, presetting a reverse-charging coefficient Rc.idx, the value of Rc.idx being 0.5; rounding up the product of the reverse-charging number and the reverse-charging coefficient to a reverse-charging value Rc.vl; defining a third marker point of the sampling point as the Rc.vl-th forward-direction point of the second marker point in the forward time direction, and denoting a time interval between the sampling point and the third marker point as a reference domain of the sampling point; and denoting the ratio of the cosine distances between the second and third marker points as a reverse-charging regression ratio Rc.rvt of the sampling points.

For any sampling point, the peak area vector of the sampling point is subtracted from that of the first sampling point in the reverse time direction thereof to obtain a step difference vector, test objects corresponding to step difference elements with values greater than zero in the step difference vector are classified as class-adding objects, otherwise, are classified as class-lossing objects, and a write sequence of each class-adding object is denoted as a class-adding sequence, the step difference element referring to any element in the step difference vector; a percentile value of the step difference element of the class-adding object in a set of step difference elements corresponding to various identical class-adding objects in the reference domain is denoted as a class-adding order value, an accumulated value of class-adding order values corresponding to all class-adding objects at the sampling point is denoted as a class-adding accumulated value Icv, and forward-direction points in the reference domain of which the class-adding accumulated value is less than the class-adding accumulated value corresponding to the current sampling point are denoted as the adaptive forward-direction points; and a quality attenuation density Pdt is calculated through the class-adding accumulated value and the reverse-charging regression ratio:

Pdt(i0)=Rc.rvtᵢ₀Σᵢ₁₌₁^{len}exp(Icvᵢ₀−Icvᵢ₁)·diff{Pmksᵢ₀,Pmksᵢ₁};
where i0 is the serial number of the sampling point, Pdt(i0) represents the quality attenuation density of the i0th sampling point, i1 is the accumulated variable, len is the number of adaptive forward-direction points corresponding to the sampling points, expo is the exponential function with the natural constant e as the base, and Icvi0 and Icvi1 are the class-adding accumulated values of the i0th sampling point and the i1^{th} adaptive forward-direction point; and
diff{ } is the Euclidean distance function, which returns the Euclidean distance between two calling sequences, Pmksi0 and Pmksi1 represent the class-adding reading sequences of the i0th sampling point and the i1^{th} adaptive forward-direction point respectively, and the class-adding reading sequence is composed of the class-adding sequences when the i0th sampling point and the i1^{th} adaptive forward-direction point correspond to various step difference elements.

Further, in step S500, the method for feeding back the circulation step where the risk of quality attenuation occurs to the manager or management system through the quality attenuation density includes: setting a risk management time period (RMT), the value of the RMT being 60 minutes; taking any sampling point as a current sampling point, and taking the RMT period in the reverse time direction of the current sampling point as a current risk management period; denoting the quality attenuation density of the current sampling point as Pdt, and denoting the average of various quality attenuation densities in the current risk management period as EPdt; presetting a variable risk identification threshold RPRt, the value thereof being 1.5; if Pdt≥RPRt×EPdt, defining that the current sampling point is at the risk of quality attenuation, and that the flavor of the aquatic products deteriorates or be loses; and constructing all sampling points at the risk of quality attenuation into a sequence and sending the sequence to a manager client.

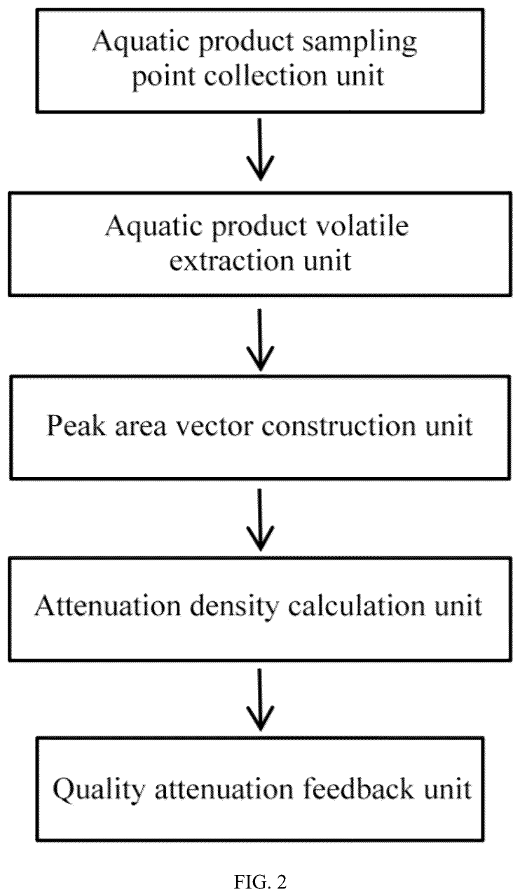

The system for tracking quality of farmed aquatic products based on lipidomic fingerprinting provided in the embodiment of the present disclosure is shown in FIG. 2, which is a structural diagram of the system for tracking quality of farmed aquatic products based on lipidomic fingerprinting of the present disclosure. The system for tracking quality of farmed aquatic products based on lipidomic fingerprinting of the embodiment includes: a processor, a memory, and a computer program stored in the memory and executable on the processor, where the processor, when executing the computer program, implements the steps in the above method for tracking quality of farmed aquatic products based on lipidomic fingerprinting.

The system includes: a memory, a processor, and a computer program stored in the memory and executable on the processor, where the processor, when executing the computer program, operates in the following system units:
an aquatic product sampling point collection unit, configured identify a farmed aquatic product circulation process and select sampling points in the aquatic product circulation process;
an aquatic product volatile extraction unit, configured to perform HS-SPME-based treatment on farmed aquatic products at each sampling point to obtain aquatic product volatile samples;
a peak area vector construction unit, configured to collect GC-MS total ion chromatogram information about the aquatic product volatile samples and construct peak area vectors;
an attenuation density calculation unit, configured to calculate a quality attenuation density according to the peak area vector of each sampling point; and
a quality attenuation feedback unit, configured to feed back to a circulation step where a risk of quality attenuation occurs to a manager or management system through the quality attenuation density.

The system for tracking quality of farmed aquatic products based on lipidomic fingerprinting is runnable in a computing device such as a desktop computer, a laptop computer, a handheld computer, or a cloud server. The runnable system may include, but is not limited to, a processors and a memory. Those skilled in the art can understand that the examples described are merely examples of a system for tracking quality of farmed aquatic products based on lipidomic fingerprinting and do not constitute a limitation on such a system. The system may include more or fewer components, or a combination of certain components, or different components. For example, the system for tracking quality of farmed aquatic products based on lipidomic fingerprinting may further include an input/output device, a network access device, and a bus.

The processor may be a central processing unit (CPU), or other general-purpose processors, digital signal processors (DSP), application-specific integrated circuits (ASIs), field-programmable gate arrays (FPGA), or other programmable logic devices, discrete gate or transistor logic devices, discrete hardware components, etc. The general-purpose processor may be a microprocessor, or may be any conventional processor. The processor is the control center of the system for tracking quality of farmed aquatic products based on lipidomic fingerprinting, and connects various parts of the entire system for tracking quality of farmed aquatic products based on lipidomic fingerprinting by means of various interfaces and lines.

The memory may be used to store the computer program and/or modules. The processor implements various functions of the system for tracking quality of farmed aquatic products based on lipidomic fingerprinting by running or executing the computer program and/or modules stored in the memory and calling the data stored in the memory. The memory may mainly include a program storage area and a data storage area, where the program storage area may store an operating system and an application program required for at least one function (such as sound playback function, image playback function, etc.); and the data storage area may store data (such as audio data, phonebook, etc.) created based on the use of a mobile phone. In addition, the memory may include a high-speed random access memory, and may also include a non-volatile memory, such as hard disk, memory, plug-in hard disk, smart media card (SMC), secure digital (SD) card, flash card, at least one disk storage device, flash memory device, or other volatile solid-state storage device.

Although the present disclosure has been described in considerable detail and particularly with regard to several of the described embodiments, it is not intended to be limited to any of these details or embodiments or any particular embodiment, thereby effectively covering the intended scope of the present disclosure. Furthermore, the present disclosure has been described above with reference to foreseeable embodiments for the purpose of providing a useful description, while non-substantial modifications to the present disclosure that have not yet been foreseen may still represent equivalent modifications to the present disclosure.

What is claimed is:

1. A method for tracking quality of farmed aquatic products based on lipidomic fingerprinting, comprising the following steps:
S100, identifying a farmed aquatic product circulation process and selecting sampling points in the aquatic product circulation process;
S200, performing HS-SPME-based treatment on farmed aquatic products at each sampling point to obtain aquatic product volatile samples;
S300, collecting GC-MS total ion chromatogram information about the aquatic product volatile samples and constructing peak area vectors;
S400, calculating a quality attenuation density according to the peak area vector of each sampling point; and
S500, feeding back a circulation step where a risk of quality attenuation occurs to a manager or management system through the quality attenuation density, wherein
a time interval formed by all sampling points is set as a sampling period, and sampling points in reverse and forward time directions of each sampling point are defined as backward-direction and forward-direction points thereof respectively; cosine distances between peak area vectors of each sampling point and all forward-direction points thereof are calculated, and the forward-direction point with the maximum cosine distance is denoted as a first marker point; the average of the cosine distances of all the forward-direction points is denoted as a forward-direction cosine distance; a first distance condition is set to be that the cosine distance is greater than the forward-direction cosine distance, and a second distance condition is set to be that at least one intermediate forward-direction point having a cosine distance less than the forward-direction cosine distance exists between the sampling point and the forward-direction point; a first marker point that satisfies the first and second distance conditions is searched in reverse time order from the sampling points and is denoted as a second marker point; the number of backward-direction points that have the same second marker point as a current sampling point in the reverse time direction of the sampling points is counted and denoted as a reverse-charging number, and a reverse-charging coefficient Rc.idx is preset, Rc.idx∈[0.3, 1]; the product of the reverse-charging number and the reverse-charging coefficient is rounded up to a reverse-charging value Rc.vl, a third marker point of the sampling point is defined as the Rc.vl-th forward-direction point of the second marker point in the forward time direction, and a time interval between the sampling point and the third marker point is denoted as a reference domain of the sampling point; and the ratio of cosine distances between the second and third marker points is denoted as a reverse-charging regression ratio Rc.rvt of the sampling points;
for any sampling point, the peak area vector of the sampling point is subtracted from that of the first sampling point in the reverse time direction thereof to obtain a step difference vector, test objects corresponding to step difference elements with values greater than zero in the step difference vector are classified as class-adding objects, otherwise, are classified as class-lossing objects, and a write sequence of each class-adding object is denoted as a class-adding sequence, the step difference element referring to any element in the step difference vector; a percentile value of the step difference element of the class-adding object in a set of step difference elements corresponding to various identical class-adding objects in the reference domain is denoted as a class-adding order value, an accumulated value of class-adding order values corresponding to all class-adding objects at the sampling point is denoted as a class-adding accumulated value Icv, and forward-direction points in the reference domain of which the class-adding accumulated value is less than the class-adding accumulated value corresponding to the current sampling point are denoted as adaptive forward-direction points; and a quality attenuation density Pdt is calculated through the class-adding accumulated value and the reverse-charging regression ratio:
Pdt(i0)=Rc.rvtᵢ₀Σᵢ₁₌₁^{len} exp(Icvᵢ₀−Icvᵢ₁)−diff{Pmksᵢ₀,Pmksᵢ₁};
wherein i0 is the serial number of the sampling point, Pdt(i0) represents the quality attenuation density of the i0^{th} sampling point, i1 is the accumulated variable, len is the number of adaptive forward-direction points corresponding to the sampling points, expo is the exponential function with the natural constant e as the base, and Icvi0 and Icvi1 are the class-adding accumulated values of the i0^{th} sampling point and the i1^{th} adaptive forward-direction point; and diff{ } is the Euclidean distance function, which returns the Euclidean distance between two calling sequences, Pmksi0 and Pmksi1 represent the class-adding reading sequences of the i0^{th} sampling point and the i1^{th} adaptive forward-direction point respectively, and the class-adding reading sequence is composed of the class-adding sequences when the i0^{th} sampling point and the i1^{th} adaptive forward-direction point correspond to various step difference elements.

2. The method for tracking quality of farmed aquatic products based on lipidomic fingerprinting according to claim 1, wherein in step S100, the method for identifying the farmed aquatic product circulation process and selecting the sampling points in the aquatic product circulation process comprises: involving an environment detection device in the farmed aquatic product circulation process, which consists of a humidity sensor and a temperature sensor; if a temperature value measured by the temperature sensor in real time is not within the temperature range of 0-4° C., or if a humidity value measured by the humidity sensor in real time is not within the humidity range of 85-95%, defining that abnormal temperature or humidity occurs at this moment; presetting a time interval GTT between sampling points to be 10-30 minutes, if no abnormal temperature or humidity occurs within the GTT interval, taking an end moment of the GTT interval as a sampling point, otherwise, recording a time point when abnormal temperature or humidity occurs as a sampling point in the time period.

3. The method for tracking quality of farmed aquatic products based on lipidomic fingerprinting according to claim 1, wherein in step S200, the method for performing HS-SPME-based treatment on the farmed aquatic products at each sampling point to obtain the aquatic product volatile samples comprises:
selecting farmed aquatic products at sampling points for sampling to obtain original samples; and collecting aquatic product volatile samples from the original samples through an HS-SPME solid-phase microextraction method, wherein in the application of the HS-SPME technology, the extraction temperature is set between 30-60° C., and the extraction time is set between 10-30 minutes.

4. The method for tracking quality of farmed aquatic products based on lipidomic fingerprinting according to claim 1, wherein in step S300, the method for collecting the GC-MS total ion chromatogram information about the aquatic product volatile samples and constructing the peak area vectors comprises: introducing the aquatic product volatile samples into a GC-MS gas chromatography-mass spectrometry instrument to obtain a total ion chromatogram, reading each aquatic product volatile in the total ion chromatogram and denoting the aquatic product volatile as a test object, reading the peak area of each test object, and constructing the peak area of each test object in the sampling point into a vector and denoting the vector as a peak area vector.

5. The method for tracking quality of farmed aquatic products based on lipidomic fingerprinting according to claim 1, wherein in step S500, the method for feeding back the circulation step where the risk of quality attenuation occurs to the manager or management system through the quality attenuation density comprises: setting a risk management time period RMT, RMT∈[60, 120] minutes; taking any sampling point as a current sampling point, and taking the RMT period in the reverse time direction of the current sampling point as a current risk management period; denoting the quality attenuation density of the current sampling point as Pdt, and denoting the average of various quality attenuation densities in the current risk management period as EPdt; presetting a variable risk identification threshold RPRt, a value range thereof being RPRt∈[1,1,2]; if Pdt>RPRt×EPdt, defining that the current sampling point is at the risk of quality attenuation, and that the flavor of the aquatic products deteriorates or loses, and constructing all sampling points at the risk of quality attenuation into a sequence and sending the sequence to a manager client.

6. A system for tracking quality of farmed aquatic products based on lipidomic fingerprinting, comprising: a processor, a memory, and a computer program stored in the memory and executable on the processor, wherein the processor, when executing the computer program, implements the steps in the method for tracking quality of farmed aquatic products based on lipidomic fingerprinting of claim 1, and the system for tracking quality of farmed aquatic products based on lipidomic fingerprinting is runnable in a computing device such as a desktop computer, a laptop computer, a handheld computer, or a cloud data center.
